(19) United States
(12) Patent Application Publication
(10) Pub. No.: US 2007/0237727 A1
(43) Pub. Date: Oct. 11, 2007

(54) Composition and method of bleaching teeth

(75) Inventor: Alan B. Matthews, Santa Maria, CA (US)

Correspondence Address: CROWELL & MORING LLP;INTELLECTUAL PROPERTY GROUP, P.O. BOX 14300, WASHINGTON, DC, 20044-4300, US

(73) Assignee: Den-Mat Corporation, Santa Maria, CA (US)

(21) Appl. No.: 11/729,929

(22) Filed: Mar. 30, 2007

Related U.S. Application Data
(60) Provisional application No. 60/787,500, filed on Mar. 31, 2006.

(51) Int. Cl. A61K 8/19

(52) U.S. Cl. 424/53

(57) ABSTRACT

Methods of using tooth bleaching gels with a high concentration of peroxide are disclosed. The present invention also provides a method for treating one or more teeth. The method includes steps of applying a desensitizing gel to one or more teeth, applying therapeutically effective amount of a tooth bleaching gel with a high concentration of peroxide to one or more teeth, and leaving the gel in contact with the one or more teeth such that the gel may whiten the tooth or teeth.

### PRIORITY STATEMENT

This application claims priority to U.S. provisional application No. 60/787,500 filed Mar. 31, 2006.

### FIELD OF THE INVENTION

The present invention relates to compositions and methods for bleaching teeth. More particularly, the present invention is directed to tooth bleaching gels with enhanced stability and that are substantially free from bleaching sensitivity.

### BACKGROUND OF THE INVENTION

White teeth have long been considered cosmetically desirable. Unfortunately, teeth almost invariably become discolored. Over time the use of certain foods and tobacco, the process of aging, diseases, trauma, medications, some congenital conditions, and environmental effects cause teeth to become discolored to varying degrees. This inevitable discoloration combined with the desire for whiter teeth have led to a high level of interest in developing compositions and methods for bleaching teeth. To this end, people have in the past relied on mechanical cleaning methods, having veneers placed over their teeth or having their teeth chemically bleached in order to achieve a desired level of tooth whiteness.

A tooth is comprised of an inner dentin layer, an outer hard, slightly porous, enamel layer and the acquired pellicle. The natural color of the tooth is opaque to translucent white or off-white. Tooth enamel is predominantly formed from inorganic material, mostly in the form of hydroxyapatite crystals and further contains approximately 5% organic material primarily in the form of collagen. The dentin layer is composed of about 20% protein, including collagen, with the balance consisting of inorganic material, predominantly hydroxyapatite crystals, similar to that found in enamel. The acquired pellicle is a proteinaceous layer on the surface of tooth enamel which reforms rapidly after an intensive tooth cleaning.

Tooth staining can generally be characterized as extrinsic or intrinsic. Staining of the acquired pellicle arises as a result of compounds such as tannins and polyphenolic compounds coming in contact with the tooth when eating, drinking or smoking. These compounds then become trapped in and tightly bound to the proteinaceous layer on the surface of the teeth. This type of staining is extrinsic. Extrinsic staining is typically removed by mechanical methods of tooth cleaning, such as brushing and/or flossing. In contrast, intrinsic staining occurs when staining compounds penetrate the enamel and even the dentin or arise from sources within the tooth. This type of staining cannot typically be addressed via mechanical methods of tooth cleaning. Chemical methods are required to remove this type of staining.

The vast majority of tooth-bleaching systems in use currently employ a form of peroxide as a bleaching agent. The efficacy of peroxide compounds in oral hygiene has been long recognized. Peroxide has been used by dental clinicians for several decades as an oral antiseptic. Such compounds have proven effective in the treatment of gingivitis, oral lesions, periodontitis, herpetic stomatitis and in combating plaque. Tooth bleaching was an observed side effect of extended contact time, thus, peroxide compounds have been utilized for oral cosmetic purposes such as tooth bleaching. The majority of tooth-bleaching compounds in use currently employ carbamide peroxide (CO(NH2)2H2O2), also called urea hydrogen peroxide, hydrogen peroxide carbamide, and perhydrol-urea, as the bleaching agent. Also, peroxide salts of the alkali or alkaline earth metals are known to be useful in bleaching teeth.

U.S. Pat. Nos. 5,098,303, 5,234,342, 5,376,006, and 5,725,843, herein incorporated by reference, teach water-based bleaching gels that contain carbomer, glycerin, and a peroxide such as hydrogen peroxide or carbamide peroxide (urea peroxide). In addition, U.S. Pat. No. 3,657,413, describes a bleaching composition that contains urea peroxide.

Further examples of tooth bleaching compositions containing peroxides can found in the disclosures of U.S. Pat. Nos. 4,839,157 and 4,405,599. These compositions included various abrasive agents such as, dicalcium phosphate, calcium carbonate, magnesium carbonate, silica or polyethylene compounds. The use of abrasive constituents in dentifrices containing peroxide compounds results irritation to both tooth and gum surfaces which is further compounded by the interaction of the peroxide composition on the abraded surfaces.

U.S. Pat. Nos. 5,098,303, 5,376,006 and 5,725,843 teach high viscosity sustained release dental compositions, such as tooth bleaching or fluoride compositions, for treating tooth surfaces. The sustained release dental compositions include a high carboxypolymethylene concentration (typically greater than 3.5%) which results in very high viscosity. The bleaching gels can contain from about 3 to about 20% carbamide peroxide, preferably about 4% to about 15% carbamide peroxide. Alternatively, the bleaching gels can contain hydrogen peroxide in a preferred range of from about 2% to about 10%.

U.S. Pat. No. 4,226,851 teaches a stable dental hygiene composition comprising a mixture of hydrogen peroxide and zinc chloride. The mixture is stabilized by the addition of water soluble vitamin E.

Ultradent, produced by Ultradent Products of South Jordan, Utah, offers a 35% hydrogen peroxide bleaching gel product called Opalescence Xtra. The package contains a prominent warning which states “REFRIGERATION REQUIRED!” Similar warnings appear in two places on the package insert. Opalescence Xtra is a gel that is red in color due to the presence of .beta.-carotene. Opalescence Xtra turns into a colorless, runny liquid in less than two weeks when stored at room temperature.

None of the aforementioned references describes the stabilization of gels with respect to both gel stability and hydrogen peroxide stability. The tooth bleaching agents described above suffer from a relatively short shelf life even when refrigerated due to the collapse of the carbomer gel, resulting in a product that is too fluid for use in the mouth. Thus, it would be desirable to develop a bleaching gel with a high amount of peroxide and improved stability.

A common side effect of dental bleaching is tooth sensitivity. Sensitivity tends to be more pronounced with bleaching compounds having higher peroxide concentrations, such as those intended for application by the dentist, only (“chairside” treatments). In most cases involving sound teeth, this sensitivity is transient, resolving itself without any lasting effect within hours to days. However in many cases, it is severe enough to prematurely terminate bleaching treatments.

Some studies that report tooth sensitivity, ranging from 13-78% of those treated with (mostly low percentage) carbamide peroxide or hydrogen peroxide gels:
Goodson, J. M., et al, “Light augments tooth whitening with peroxide,” JADA, February 2003, Vol 134 (2): 167-175. Cited 20-21.7% of subjects bleached had moderate to greatly increased tooth sensitivity.
Haywood, V. B., et al, “Tray delivery of potassium nitrate-fluoride to reduce bleaching sensitivity,” February 2001, Quintessence Int., Vol 32 (2): 105-9. Study cited 16 of 30 patients (53.3%) experienced tooth sensitivity.
Gerlach, R. W., et al, “Comparative response of whitening strips to a low peroxide and potassium nitrate bleaching gel,” Am J Dent. September 2002, 15 Spec No: 19A-23A. Study reported 13% of subjects using the hydrogen peroxide strips reported sensitivity and 22% of the subjects using carbamide peroxide-potassium nitrate tray system had sensitivity.
Leonard, R. H. Jr., et al, “Desensitizing agent efficacy during whitening in an at-risk population,” J Esthet Restor Dent, 2004, 16 (1): 49-55. Reported that 78% of a placebo group, treated with 10% carbamide peroxide solution without sodium fluoride and KNO3, experienced sensitivity.
Browning, W. D., “Safety and efficacy of a nightguard bleaching agent containing sodium fluoride and potassium nitrate,” Quintessence Int., October 2004, 35 (9): 693-8. 36% of subjects reported sensitivity during the active whitening phase of the study.

Tam, L., “Clinical trial of three 10% carbamide peroxide bleaching products,” J Can Dent Assoc, April 1999, 65 (4): 201-5. 64% of the patients reported sensitivity.

Additionally, the following study was cited in U.S. Pat. No. 6,458,340, Desensitizing bleaching gel, Ibsen, R., et al:
Haywood et al. J. Amer. Dent. Assn. 125:1219-1226 Effectiveness, Side Effects and Long Term Status of Nightguard Vital Bleaching (Sensitivity During or After Bleaching) September 1994.

None of the above disclosed tooth bleaching compositions address the issue of transient dental sensitivity that might arise from a bleaching agent with a high concentration of peroxide.

The instant invention solves these problems by providing a method of using a bleaching composition with a high concentration of peroxide that reduces instances of dental sensitivity.

### SUMMARY OF THE INVENTION

This invention contemplates methods and compositions for bleaching teeth and improving overall oral hygiene.

This invention also contemplates tooth bleaching methods that reduce instances of dental sensitivity.

In one aspect, the present invention provides a method for using a composition for bleaching teeth that comprises a high concentration of peroxide.

A further aspect of the invention provides a method for using a composition for bleaching teeth that comprises a high concentration of peroxide, Laponite®, and 0.03 micron synthetic silica.

In a further aspect of the invention, the present invention provides a method for treating one or more teeth. The method includes steps of applying a thin layer of desensitizing gel to one or more teeth, applying a therapeutically effective amount of a stable bleaching composition directly on top of the layer of desensitizing gel and leaving the bleaching composition in contact with the one or more teeth such that the bleaching composition may whiten the tooth or teeth.

In another aspect of the invention, the present invention provides a method of treating one or more teeth. The method includes steps of applying a thin layer of desensitizing gel to one or more teeth, applying a therapeutically effective amount of a stable bleaching composition directly on top of the layer of desensitizing gel and exposing the treated teeth one at a time to high energy UV-free visible light from a Plasma Arc light source. The preferred light source is a Sapphire™ PAC Curing Light (Den-Mat® Corporation, Santa Maria, Calif. USA). Alternately, the Sapphire Whitening Crystal (Den-Mat® Corporation, Santa Maria, Calif. USA; U.S. Pat. No. 6,733,290 B2) can be employed to simultaneously expose both arches of treated teeth.

In another aspect of the invention, the desensitizer gel of the present invention can be adjusted to a high pH with suitable alkaline ingredients. Then, this method includes steps of applying a thin layer of the desensitizing gel to one or more teeth and applying a therapeutically effective amount of a stable bleaching composition directly over the high-pH desensitizing gel. This embodiment has the advantage of accelerating the release of peroxide radicals that will improve the present invention's bleaching effectiveness while also protecting the patient's teeth from sensitivity. As above, the efficacy of this embodiment can be further enhanced by exposure to high-energy visible light from the Sapphire PAC Light, either one tooth at a time or simultaneously with the Sapphire Whitening Crystal.

In another aspect, the present invention provides a dental bleaching system comprising: a layer of an aqueous, desensitizing gel comprising potassium nitrate; and a layer of a dental bleaching gel comprising Laponite® and an element selected from the group consisting of: hydrogen peroxide and compounds that yield hydrogen peroxide in an aqueous environment. The bleaching gel may further comprise a microfine silica, preferably a 0.03 micron silica. The desensitizing gel may also be adjusted to a high pH.

In still another aspect, the present invention provides a desensitizing gel comprising a gelling agent, a strong base and potassium nitrate wherein when the desensitizing gel is applied to a patient's tooth and covered with a peroxide dental bleaching gel, the desensitizing gel accelerates the bleaching action of the bleaching gel and desensitizes the patient to the bleaching action.

### DETAILED DESCRIPTION OF THE INVENTION

For simplicity and illustrative purposes, the principles of the present invention are described by referring to various exemplary embodiments thereof. Although the preferred embodiments of the invention are particularly disclosed herein, one of ordinary skill in the art will readily recognize that the same principles are equally applicable to, and can be implicated in other compositions and methods, and that any such variation would be within such modifications that do not part from the scope of the present invention. Before explaining the disclosed embodiments of the present invention in detail, it is to be understood that the invention is not limited in its application to the details of any particular embodiment shown, since of course the invention is capable of other embodiments. The terminology used herein is for the purpose of description and not of limitation. Further, although certain methods are described with reference to certain steps that are presented herein in certain order, in many instances, these steps may be performed in any order as may be appreciated by one skilled in the art, and the methods are not limited to the particular arrangement of steps disclosed herein.

As stated, the instant invention is directed to tooth bleaching methods using gels that contain hydrogen peroxide and that are stable at room temperature for an extended period of time. These gels may comprise: (i) a solvent; (ii) a thickening agent; (iii) bleaching agent; (iv) stabilizing agent; and, optionally, (v) a gelling agent, (vi) a desensitizing agent, (vii) alkaline ingredient to increase gels' pH to further accelerate the bleaching process. One of the consequences of using such gels is that in some instances the use of these gels results in dental sensitivity. The present invention addresses this issue.

Tooth bleaching gels to be used in the instant invention will include at least one solvent. Examples of solvents that may be used in the invention include, but are not limited to, water, glycerin, propylene glycol, polyethylene glycol, or a variety of other compounds suitable for use as solvents for oral applications.

The gelling agent used in gels of the instant invention may be present in an amount ranging from 5 to 20%. In the past, practitioners have relied on carbomer as a preferred gelling agent. The co-pending application of Joseph Campenele and W R Glace, filed concurrently herewith and which claims priority to application No. 60/787,501 filed Mar. 31, 2006 and incorporated herein by reference discloses that by replacing carbomer with a mixture of Laponite® (Rockwood Clay Additives GMBH) and 0.03 micron synthetic silica the shelf life of the resulting tooth bleaching gel is significantly extended. Laponite® is a highly-purified, synthetic, patented colloidal clay, that imparts viscosity and suspension properties to the gel, thereby enhancing its stability. There are many grades of Laponite® offered for applications ranging from industrial, surface coatings, agricultural, paper, household products and personal care products. These products can be divided into two general types, however: gel-forming grades and sol-forming grades. The difference between the two types is that the sol-forming grades have had tetrasodium pyrophosphate added as a dispersant to the gel-forming grades. Use of the sol-forming grades of Laponite® permit complete dispersion and incorporation of ingredients prior to gel formation. In the instant invention, gel formation is enhanced by the addition of an acid-form of pyrophosphate such as sodium acid pyrophosphate and shifts the gel's pH lower to further improve the gel's peroxide stability. The present invention is not limited, however, to the sol-forming grades; they do constitute the preferred embodiment.

The bleaching agent utilized in the gel is hydrogen peroxide present in an amount ranging from 3 to 50%, preferably 20 to 50%, more preferably 25-40%, and most preferably 25% by weight of the aqueous gel. Higher amounts of bleaching agent are preferred so that the gel may serve as a “fast acting bleaching gel” capable of bleaching teeth with only one or two applications during a single visit to the dental office.

The bleaching agent may be selected from hydrogen peroxide (H₂O₂) or any compound that yields hydrogen peroxide when placed in an aqueous medium. For example, carbamide peroxide (CO(NH₂)₂H₂O₂) generates hydrogen peroxide when placed in water. Other names for carbamide peroxide include urea peroxide, urea hydrogen peroxide, hydrogen peroxide carbamide, and perhydrol urea.

The stabilizing agent utilized in the gel is present in an amount ranging from 0.05 to 1% by weight of the aqueous gel. The stabilizing agent is selected from aminocarboxylic acids and salts thereof. Preferred stabilizers are selected from aminocarboxylic acids and alkali and/or alkali earth metal salts thereof. Suitable aminocarboxylic acids include trans-1,2-cyclohexylene dinitrilotetraacetic acid (CDTA), ethylenediamine tetraacetic acid (EDTA), N-(2-hydroxyethyl)ethylenediamine triacetic acid (HEDTA), Nitrilotriacetic acid (NTA), diethylene triamine pentaacetic acid (DTPA), triethylene tetraamine hexaacetic acid (TTHA), and ethyleneglycol bis (2-aminoethylether)tetraacetic acid (GEDTA). The most preferred stabilizers include CDTA, CaNa₂ EDTA, Na₂ EDTA, Na₄ EDTA, HEDTA, and Na₃ HEDTA.

The combination of the above ingredients provides a gel with a high concentration of hydrogen peroxide and that maintains the integrity of the gel for extended periods of time, even at room temperature. In addition, there is a reduction in hydrogen peroxide decomposition during storage.

It may also be desirable to include other ingredients in the composition of the present invention. Such ingredients may include a flavor enhancing agent such as peppermint or spearmint oil, or any other flavor agent known in the art. Other ingredients may also include a sweetener such as sodium saccharin, acesulfame potassium, xylitol, sucralose, or any other known sweetener. In addition to the previously mentioned ingredients, the composition of the present invention may include a solubilizing or emulsifying agent. Such agents include any known solubilizing agent suitable for oral applications.

The end result is that the aqueous gels can now be produced that have commercially viable shelf-lives at room temperature. Thus, constant refrigeration, which is both expensive and inconvenient, especially during transit, is no longer necessary.

The dental bleaching gel of the instant invention can be applied to the teeth in a number of ways. In example, the gel can be applied to the teeth using a brush, syringe, tray, or any other application means.

In a typical treatment process, the soft tissues surrounding the teeth are first covered with a protecting device, e.g. a ligated rubber dam or polymerized dental resin such as Paint-On Dental Dam (Den-Mat® Corporation, Santa Maria, Calif. USA). This is important because the more hydrogen peroxide a dental bleaching gel contains, the more likely it is to burn the soft tissue upon contact. Dental bleaching gels containing at least 30% by weight hydrogen peroxide will immediately burn any soft tissue they contact, quickly turning the tissue white. Next a brush, needle, or some other delivery system is utilized to place the dental bleaching gel described above in contact with the teeth one wishes to bleach. Most patients only request treatment on the labial surfaces of the 6 to 8 front teeth which show most prominently when one smiles. The dental bleaching gel is then allowed to remain in contact with the teeth for a period of time ranging anywhere from 5 minutes to one hour. One of skill in the art will recognize that the bleaching effect of any dental bleaching gel is directly proportional to this residence time.

The bleaching effect of the hydrogen peroxide in a given period of time may be amplified by applying a powerful visible light, a heat lamp or laser light to the dental bleaching gel once it is in place on the teeth. The heat and light serve to increase the rate of bleaching of the hydrogen peroxide, providing a shorter period of time for bleaching the teeth. Upon completion of the treatment, the gel is removed with a gauze pad or some other means. The patient's mouth is then thoroughly cleaned with water and suction.

One of the consequences of dental bleaching in some instances is transient dental sensitivity. Higher levels of oxidizing agents lead to more frequent instances of dental sensitivity. Thus, it is desirable to protect the teeth from sensitivity caused by high levels of oxidizing agents by treating the teeth with a desensitizing agent. Similarly, patients and dentists both prefer that a bleaching treatment require less, rather than more, time. It is well-known in the dental profession and common to find mention in references to peroxide chemistry, that when one elevates the pH of a peroxide solution, it accelerates its decomposition. Of course, the higher the pH of a peroxide solution, the less stable it is. And, unstable peroxide solutions are undesirable in storage as they tend to disrupt typical gel matrices and lead to liquification of the gel and, as importantly, evolve tremendous amounts of gas that can swell and break tubes or forcibly discharge plungers from syringes. However the Laponite® microfine silica mixture utilized in the gel described herein results in a firm, smooth gel with a pH that is initially neutral (pH about 6.5) but drifts within hours upwards to about 8.0. But even at this pH, which would be unsuitable for a carbomer-thickened gel due to peroxide instability, the Laponite® gel is stable and does not evolve gas.

In addition, the Laponite®-microfine silica mixture may have a single drawback in that because of the way Laponite® forms gels, it is subject to dissociation in the presence of high ionic mixtures. Thus, it would not be possible to create a single high-peroxide mixture containing both Laponite®-microfine silica and the preferred dental desensitizer, potassium nitrate. For the same reason, an alternate dental desensitizer—sodium fluoride at about 1.1% by weight—has been found to also disrupt the Laponite® gel matrix.

In order to solve this problem and provide both an accelerant and desensitizer to the bleaching system, it was decided that a high-pH carbomer gel that contained the desensitizer potassium nitrate would be used. There would be no issue with incorporating potassium nitrate in a high pH gel that could be brushed-on in a thin layer to coat the teeth prior to application of the bleaching gel mixture. The potassium nitrate would act as a dental desensitizer while the elevated pH would act to accelerate the bleaching process. Since both the accelerant-desensitizer gel and the bleaching gel layer are aqueous, and since the act of brushing the bleaching gel onto the teeth would also act to mix the accelerant-desensitizer and the bleaching gels together, peroxide would rapidly diffuse onto the teeth. The desensitizing gel comprises 1-10 w % potassium nitrate and more preferably 5w % potassium nitrate. A desensitizing gel of this invention would comprise, e.g., 75-85 wt % water; 0-5 wt % carbomer; and 1-10 wt % potassium nitrate; preferably 79.6 wt % water; 14.2 wt % carbomer; and 5.0 wt % potassium nitrate. A desensitizing gel of this invention may also comprise, e.g., 75-85 wt % water; 10-20 wt % potassium phosphate (97% by volume); 0-5 wt % carbomer; and 1-10 wt % potassium nitrate; preferably 79.6 wt % water; 14.2 wt % potassium phosphate (97% by volume); 1.2 wt % carbomer; and 5.0 wt % potassium nitrate.

It has been found during clinical evaluation that the application of a thin (approximately 0.5 mm) layer of the desensitizing-accelerant gel followed by a thicker (1.0 to 1.5 mm) layer of the bleaching gel achieves optimal bleaching without sensitivity. Additionally, clinical evaluation also established that the use of a 25% hydrogen peroxide bleaching gel was as effective under the methods of use described herein as a 32% variant. Also, by following this method, neither the 25 or 32% versions applied over the accelerant-desensitizing gel resulted in sensitivity, but patients treated with another bleaching product without desensitizer reported sensitivity.

There is no evidence to suggest that the simultaneous application of a mixture of the accelerant-desensitizer and the bleaching gel would achieve the same levels of bleaching without sensitivity; thus, use of dual-chamber syringes having 1:4 (or similar) mixing ratios, while contemplated, were not evaluated clinically.

### EXAMPLE 1

A stable gel composition suitable for the instant invention may have the following ingredients:

| INGREDIENT | wt % |
|---|---|
| water | 7.833 |
| glycerin | 7.833 |
| Laponite ® | 3.00 |
| 50% H₂O₂ | 71.988 |
| Sodium Flouride | 1.100 |
| EDTA | 0.665 |
| Sodium acid pyrophosphate | 2.000 |
| Flavor | 1.100 |
| Microfine silica | 4.483 |

### EXAMPLE 2

A suitable Accelerant-Desensitizer gel may have the following ingredients:

| INGREDIENT | % w/w |
|---|---|
| Water | 78.400 |
| Potassium phosphate 97% | 14.100 |
| Carbopol ETD 2020 | 2.500 |
| Potassium nitrate USP | 5.000 |

### EXAMPLE 3

The steps below describe one embodiment of the instant invention.

1. Prophy teeth to remove calculus and extrinsic stains before beginning the whitening treatment.

2. Take “before” photographs and record tooth color using a shade guide arranged in bleaching order.

3. Insert cheek retractors. Have the patient bite down and rest tongue on a tongue block.

4. Air-dry the gingival tissue and teeth.

5. Using the dispensing tip or disposable brush, apply Den-Mat® Paint-On Dental Dam to the gingiva to isolate gums around the selected teeth. Overlap the dental dam onto the gingival tissue of adjacent teeth about 0.5 mm and light-cure for 1-2 seconds per tip width with a Sapphire™ Power Arc Curing (PAC) Light.

6. Once the Paint-On Dental Dam has been cured, apply Den-Mat Moisturizing Gel to the lips and mucosa, applying well beyond the vermillion border.

7. Remove the tip from the end of the Sapphire Desensitizer recappable syringe and dispense the Desensitizer in a very thin (about 0.5 mm) layer on the labial surface of the teeth. Distribute the gel evenly using the brush applicator provided in the kit. Firmly recap the Desensitizer syringe.

8. Immediately remove the tip from the Sapphire Whitening syringe and apply a 1-1.5 mm layer directly onto the labial surface of the teeth. Use the brush applicator to distribute the gel evenly over the teeth treated in Step 7 with the Sapphire Desensitizer Gel. Monitor the patient's comfort. Allow the treatment to remain on the patient's teeth for twenty (20) minutes, adding additional gel if needed. Place the cap back on the syringe tip to protect remaining contents.

9. At the end of the twenty minute session, vacuum gel from the teeth, wipe with gauze and rinse the teeth while suctioning. Avoid splatter.

10. Peel away the Dental Dam and evaluate the results with the patient. Some patients with heavy discoloration or areas of unseen hypocalcification may need a second 20-minute session to achieve desired bleaching results. If a second session is indicated, allow approximately five (5) minutes before resuming the treatment beginning with Step 3.

11. Use a shade guide arranged in bleaching order to compare the color of the whitened teeth to the original shade recorded in Step 2 and take ‘after’ photographs.

### EXAMPLE 4

Bleaching can be further enhanced by using the Den-Mat's Sapphire™ PAC Light

1. Perform Steps 1-8 up to the application of the Whitening Gel over the teeth treated with the Desensitizer Gel.

2. Install the Sapphire Whitening Crystal in place of the curing tip on the end of the Sapphire Light pistol.

3. Position the Whitening Crystal close to, and directly in front of the teeth, using a combination of the articulating arm and chair adjustments

Center the Whitening Crystal ¼ to ½ inch from the teeth within the cheek retractor with the outer edges touching (if possible).

4. Press the Optional button on the Sapphire Light keypad, then press 2 to select bleaching mode.

The letters bL (bleach) will appear in the Program window, the number 60 will appear in the seconds window and 60 minutes will appear on the pistol handle.

5. Press the Start or pistol activator button to start the bleaching process.

The number 60 (minutes) appears in the Program window and seconds begin to count down in the Seconds window. Press the Start button when the number has counted down to 40 (minutes); that is, when 20 minutes have elapsed.

6. Monitor the patient's comfort and the alignment of the Whitening Crystal during the entire procedure. Check the gel at 5 minute intervals. At these 5-minute periods:

Pause the light by pressing the Start or pistol activator button one time (remaining time will be retained). Move the crystal away from the teeth.

Do not remove the gel. Agitate and moisten the gel using the brush applicator to add additional gel only if needed.

Reposition the Crystal and press the Start or pistol activator button one time to resume the procedure.

At the end of the 20-minute exposure time, press the Optional button once to cancel the 40 minutes remaining on the original 60 minute program.

7. Peel away the Dental Dam.

8. Using the bleaching ordered shade guide, compare the color of the bleached teeth to the original record in Step 2 (above). Take ‘after’ photographs.

While the invention has been described with reference to certain exemplary embodiments thereof, those skilled in the art may make various modifications to the described embodiments of the invention without departing from the scope of the invention. The terms and descriptions used herein are set forth by way of illustration only and are not meant as limitations. In particular, although the present invention has been described by way of examples, a variety of compositions and methods would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

What is claimed is:

1. A method of dental treatment comprising:
providing a protecting device to at least a portion of at least one tooth;
providing a layer of an aqueous, basic desensitizing gel to at least a portion of at least one tooth not covered by the protecting device, wherein the desensitizing gel comprises potassium nitrate; and
providing a layer of a dental bleaching gel over the layer of desensitizing gel, wherein the bleaching gel comprises Laponite®.

2. The method of claim 1, wherein the desensitizing gel comprises:
water;
potassium phosphate;
a gelling agent; and
potassium nitrate.

3. The method of claim 2, wherein the desensitizing gel comprises:
75-85 wt % water;
10-20 wt % potassium phosphate (97% by volume);
0-5 wt % carbomer; and
1-10 wt % potassium nitrate.

4. The method of claim 3, wherein the desensitizing gel comprises:
79.6 wt % water;
14.2 wt % potassium phosphate (97% by volume);
1.2 wt % carbomer; and
5.0 wt % potassium nitrate.

5. The method of claim 1, wherein the dental bleaching gel comprises:
a solvent;
a gelling agent;
a bleaching agent; and
a stabilizing agent,
wherein the gelling agent comprises Laponite®.

6. The method of claim 5, wherein the gelling agent comprises Laponite® and 0.03 micron synthetic silica.

7. The method of claim 5, wherein the solvent comprises at least one element selected from the group consisting of: water, glycerin, propylene glycol, and polyethylene glycol.

8. The method of claim 5, wherein the bleaching agent comprises a peroxide.

9. The method of claim 8, wherein the bleaching agent comprises at least one element selected from the group consisting of: hydrogen peroxide and carbamide peroxide.

10. The method of claim 5, wherein the stabilizing agent comprises at least one element selected from the group consisting of: aminocarboxylic acids and aminocarboxylic acid salts.

11. The method of claim 10, wherein the stabilizing agent comprises at least one element selected from the group consisting of: aminocarboxylic acids and alkali metal aminocarboxylic acid salts, and alkaline earth metal aminocarboxylic acid salts.

12. The method of claim 11, wherein the stabilizing agent comprises at least one element selected from the group consisting of: trans-1,2-cyclohexylene dinitrilotetraacetic acid (CDTA), ethylenediamine tetraacetic acid (EDTA), N-(2-hydroxyethyl)ethylenediamine triacetic acid (HEDTA), Nitrilotriacetic acid (NTA), diethylene triamine pentaacetic acid (DTPA), triethylene tetraamine hexaacetic acid (TTHA), and ethyleneglycol bis (2-aminoethylether)tetraacetic acid (GEDTA).

13. The method of claim 12, wherein the stabilizing agent comprises at least one element selected from the group consisting of: CDTA, CaNa₂EDTA, Na₂EDTA, Na₄EDTA, HEDTA, and Na₃HEDTA.

14. The method of claim 5, wherein the dental bleaching gel further comprises an agent that initiates gel formation.

15. The method of claim 5, wherein the gel-initiation agent is sodium acid pyrophosphate.

16. The method of claim 5, wherein the gelling agent is present in an amount of between 2 and 5 wt %.

17. The method of claim 5, wherein the bleaching agent is present in an amount of between 3 and 50 wt %.

18. The method of claim 17, wherein the bleaching agent is present in an amount of between 20 and 50 wt %.

19. The method of claim 18, wherein the bleaching agent is present in an amount of between 30 and 40 wt %.

20. The method of claim 19, wherein the bleaching agent is present in an amount of 25 wt %.

21. The method of claim 5, wherein the stabilizing agent is present in an amount of between 0.05 and 1 wt %.

22. The method of claim 5, wherein the dental bleaching gel further comprises at least one of the elements selected from the group consisting of: flavor enhancing agents, sweeteners, and solubilizing agents.

23. The method of claim 1, further comprising providing electromagnetic energy to the dental bleaching gel after the bleaching gel is provided over the desensitizing gel.

24. An aqueous, basic desensitizing gel comprising:
a gelling agent;
a strong base; and
potassium nitrate,
wherein when the desensitizing gel is applied to a patient's tooth and covered with a peroxide dental bleaching gel, the desensitizing gel accelerates the bleaching action of the bleaching gel and desensitizes the patient to the bleaching action.

25. The desensitizing gel of claim 24, wherein strong base comprises potassium phosphate.

26. The desensitizing gel of claim 24, wherein the potassium nitrate is present in an amount of between 1 and 10 wt %.

27. The desensitizing gel of claim 26, wherein the potassium nitrate is present in an amount of 5 wt %.

28. A dental bleaching system comprising:
a layer of an aqueous, basic desensitizing gel comprising potassium nitrate; and
a layer of a dental bleaching gel comprising Laponite® and an element selected from the group consisting of: hydrogen peroxide and compounds that yield hydrogen peroxide in an aqueous environment.

29. The dental bleaching system of claim 28, wherein when the system is provided to a patient's tooth, the desensitizing gel accelerates the bleaching action of the bleaching gel and desensitizes a patient to the bleaching action.

30. The method of claim 3, wherein the layer of desensitizing gel is about 0.5 mm thick.

31. The method of claim 1, wherein the desensitizing gel comprises:
water;
a gelling agent; and
potassium nitrate.
